(19) United States
(12) Patent Application Publication
(10) Pub. No.: US 2026/0102141 A1
(43) Pub. Date: Apr. 16, 2026

(54) System for Bedside Cardiac Ultrasonic Monitoring

(71) Applicants: Lazaro E. Hernandez, Cooper City, FL (US); Mathias Gunnar Berg-Johansen, Sydney (AU); Jason Joseph Ryan, Sydney (AU)

(72) Inventors: Lazaro E. Hernandez, Cooper City, FL (US); Mathias Gunnar Berg-Johansen, Sydney (AU); Jason Joseph Ryan, Sydney (AU)

(21) Appl. No.: 19/375,106

(22) Filed: Oct. 30, 2025

Related U.S. Application Data
(63) Continuation-in-part of application No. 19/275,257, filed on Jul. 21, 2025, now Pat. No. 12,544,036.
Application No. 19/275,257 is a continuation-in-part of application No. 19/213,833, filed on May 20, 2025.
Application No. 19/213,833 is a continuation-in-part of application No. 29/983,303, filed on Jan. 9, 2025, now Pat. No. D1076107.
Application No. 29/983,303 is a continuation-in-part of application No. 29/973,198, filed on Nov. 14, 2024, now Pat. No. D1076105.
Application No. 29/973,198 is a continuation-in-part of application No. 29/954,765, filed on Jul. 29, 2024, now Pat. No. D1055291.
Application No. 29/954,765 is a continuation-in-part of application No. 18/651,196, filed on Apr. 30, 2024, now Pat. No. 12,178,656.
Application No. 18/651,196 is a continuation-in-part of application No. 18/471,150, filed on Sep. 20, 2023, now Pat. No. 12,290,407.
Application No. 18/471,150 is a continuation-in-part of application No. 17/935,505, filed on Sep. 26, 2022, now Pat. No. 11,957,505.
Application No. 19/213,833 is a continuation-in-part of application No. 29/983,268, filed on Jan. 9, 2025, now Pat. No. D1076106.
Application No. 29/983,268 is a continuation-in-part of application No. 29/973,198, filed on Nov. 14, 2024, now Pat. No. D1076105.
Application No. 29/973,198 is a continuation-in-part of application No. 29/954,765, filed on Jul. 29, 2024, now Pat. No. D1055291.
Application No. 29/954,765 is a continuation-in-part of application No. 18/651,196, filed on Apr. 30, 2024, now Pat. No. 12,178,656.
Application No. 18/651,196 is a continuation-in-part of application No. 18/471,150, filed on Sep. 20, 2023, now Pat. No. 12,290,407.
Application No. 18/471,150 is a continuation-in-part of application No. 17/935,505, filed on Sep. 26, 2022, now Pat. No. 11,957,505.
Application No. 17/935,505 is a continuation-in-part of application No. 17/210,013, filed on Mar. 23, 2021, now Pat. No. 11,457,889.
(60) Provisional application No. 63/141,465, filed on Jan. 25, 2021.
(60) Provisional application No. 63/739,086, filed on Dec. 26, 2024.

(51) Int. Cl. A61B 8/00; A61B 8/08

(52) U.S. Cl. CPC A61B 8/4427; A61B 8/0883; A61B 8/4483; A61B 8/462; A61B 8/465; A61B 8/56

(57) ABSTRACT

A system for bedside cardiac ultrasound monitoring enables the bedside monitoring of a cardiac ultrasound in a hospital setting. The system includes an ultrasonic transducer, a transducer housing, a bedside display system, an interface, at least one data/power cable, and a ratchet mechanism. The interface enables the removable attachment of the transducer housing to a patient's body. The transducer housing protects the ultrasonic transducer. The ratchet mechanism facilitates the adjustable coupling of the transducer housing to the interface. The ultrasonic transducer performs the cardiac ultrasound. The at least one data/power cable directly connects the ultrasonic transducer to a display controller and a display power source of the bedside display system. The display controller processes data captured by the ultrasonic transducer. The display power source provides the necessary electrical power to the system. A display housing of the bedside display system encloses the display power source and the display controller.

The current application is a continuation-in-part (CIP) application of the U.S. non-provisional utility application Ser. No. 19/275,257 filed on Jul. 21, 2025. The U.S. non-provisional utility application Ser. No. 19/275,257 is a CIP application of the U.S. non-provisional utility application Ser. No. 19/213,833 filed on Feb. 20, 2025. The U.S. non-provisional utility application Ser. No. 19/213,833 is a CIP application of the U.S. design application Ser. No. 29/983,303 filed on Jan. 9, 2025. The U.S. design application Ser. No. 29/983,303 is a CIP application of the U.S. design application Ser. No. 29/973,198 filed on Nov. 14, 2024. The U.S. design application Ser. No. 29/973,198 is a CIP application of the U.S. design application Ser. No. 29/954,765 filed on Jul. 29, 2024. The U.S. design application Ser. No. 29/954,765 is a CIP application of the U.S. non-provisional utility application Ser. No. 18/651,196 filed on Apr. 30, 2024. The U.S. non-provisional utility application Ser. No. 18/651,196 is a CIP application of the U.S. non-provisional utility application Ser. No. 18/471,150 filed on Sep. 20, 2023. The U.S. non-provisional utility application Ser. No. 18/471,150 is a CIP application of the U.S. non-provisional utility application Ser. No. 17/935,505 filed on Sep. 26, 2022. The U.S. non-provisional utility application Ser. No. 17/935,505 is a CIP application of the U.S. non-provisional utility application Ser. No. 17/210,013 filed on Mar. 23, 2021.

The current application is also a CIP application of the U.S. non-provisional utility application Ser. No. 19/275,257 filed on Jul. 21, 2025. The U.S. non-provisional utility application Ser. No. 19/275,257 is a CIP application of the U.S. non-provisional utility application Ser. No. 19/213,833 filed on Feb. 20, 2025. The U.S. non-provisional utility application Ser. No. 19/213,833 is a CIP application of the U.S. design application Ser. No. 29/983,268 filed on Jan. 9, 2025. The U.S. design application Ser. No. 29/983,268 is a CIP application of the U.S. design application Ser. No. 29/973,198 filed on Nov. 14, 2024. The U.S. design application Ser. No. 29/973,198 is a CIP application of the U.S. design application Ser. No. 29/954,765 filed on Jul. 29, 2024. The U.S. design application Ser. No. 29/954,765 is a CIP application of the U.S. non-provisional utility application Ser. No. 18/651,196 filed on Apr. 30, 2024. The U.S. non-provisional utility application Ser. No. 18/651,196 is a CIP application of the U.S. non-provisional utility application Ser. No. 18/471,150 filed on Sep. 20, 2023. The U.S. non-provisional utility application Ser. No. 18/471,150 is a CIP application of the U.S. non-provisional utility application Ser. No. 17/935,505 filed on Sep. 26, 2022. The U.S. non-provisional utility application Ser. No. 17/935,505 is a CIP application of the U.S. non-provisional utility application Ser. No. 17/210,013 filed on Mar. 23, 2021.

The current application is also a CIP application of the U.S. non-provisional utility application Ser. No. 19/275,257 filed on Jul. 21, 2025. The U.S. non-provisional utility application Ser. No. 19/275,257 claims a priority to the U.S. provisional patent application Ser. No. 63/739,086 filed on Dec. 26, 2024.

### FIELD OF THE INVENTION

The present invention generally relates to telemedicine and stationary medical devices. More specifically, the present invention discloses novel means of monitoring a cardiac ultrasound being performed at the bedside of a patient.

### BACKGROUND OF THE INVENTION

In general, cardiac ultrasounds, also known as echocardiograms, are non-invasive medical examination procedures where the heart and nearby blood vessels are monitored to detect cardiac issues in a patient. Cardiac ultrasounds are often performed in a medical facility due to the different medical instruments that are necessary to perform the procedure. Such medical instruments are difficult to move around a medical facility due to their weight and their cumbersome configuration. In addition, limiting cardiac ultrasounds to medical facilities can also limit the effectiveness of patient care if the healthcare provider requires periodic cardiac ultrasounds.

Therefore, an objective of the present invention is to provide a system for bedside cardiac ultrasound monitoring with a lightweight, less cumbersome configuration. The present invention allows a healthcare provider to review and interpret cardiac ultrasound images in real time. Another objective of the present invention is to provide a system that can be easily operated by a caregiver during the medical procedure. The present invention can readily fasten examination instruments to a patient's body without using specialized tools or assistance from a person with technical expertise. Additional features and benefits of the present invention are further discussed in the sections below.

### SUMMARY OF THE INVENTION

The present invention discloses a system for bedside cardiac ultrasound monitoring. The present invention enables healthcare providers to monitor a cardiac ultrasound being performed on a patient in a hospital setting or another kind of stationary medical facility. In the preferred embodiment, the present invention includes, but is not limited to, an ultrasonic transducer, an interface, and a data acquisition unit. The interface corresponds to the skin interface attached to the patient's body adjacent to the target examination area. The ultrasonic transducer is a hands free, portable transducer that can perform the cardiac ultrasound. The data acquisition unit corresponds to a bedside computing device that processes the data signals from the ultrasonic transducer. After the interface is placed and secured by the healthcare provider in the desired position, a medical provider is able to readily monitor the patient's echocardiogram at their bedside.

Further, the present invention may use a software application that can be installed on a patient's bedside computerized monitor to control the operation of the data acquisition unit and the ultrasonic transducer. The patient's bedside computerized monitor may have wireless capabilities and an Internet connection so that the collected data can be relayed to the healthcare database via its Internet connection.

Further, the present invention enables a process of recording a small ultrasonic loop for a period of time (e.g., 3 to 5 seconds). The recorded ultrasonic loop is then relayed to the corresponding healthcare database via the patient's bedside computerized device with the installed software application for bedside examination by an in-house healthcare provider.

### BRIEF DESCRIPTION OF THE DRAWINGS

FIG. 1 is a top-front-left perspective view of the system of the present invention.

FIG. 2 is a top-front-left perspective view of the system of the present invention, wherein the transducer housing and the interface are shown.

FIG. 3 is a bottom-rear-right perspective view of the system of the present invention, wherein the transducer housing and the interface are shown.

FIG. 4 is a left view of the system of the present invention, wherein the ratchet mechanism is shown engaged.

FIG. 5 is a left view of the system of the present invention, wherein the ratchet mechanism is shown partially disengaged.

FIG. 6 is a left view of the system of the present invention, wherein the ratchet mechanism is shown fully disengaged.

FIG. 7 is a left view of the system of the present invention, wherein the transducer housing is shown removed from the interface.

FIG. 8 is a top-front-left perspective view of the transducer housing of the system of the present invention.

FIG. 9 is a bottom-rear-right perspective view of the transducer housing of the system of the present invention.

FIG. 10 is a front view of the transducer housing of the system of the present invention.

FIG. 11 is a top-front-left perspective view of the interface of the system of the present invention.

FIG. 12 is a bottom-rear-right perspective view of the interface of the system of the present invention.

FIG. 13 is a left view of the interface of the system of the present invention, wherein the lower base face of the interface is shown with adhesive.

FIG. 14 is a box diagram showing the electrical connections and the electronic connections of the system of the present invention, wherein the electrical connections are shown in solid lines, and wherein the electronic connections are shown in dashed lines.

FIG. 15 is a schematic cross-sectional view of the ultrasonic transducer of the system of the present invention.

FIG. 16 is a top-front-left perspective view of the system of the present invention, wherein the bedside display system is shown with a swivel arm and a wall mount.

FIG. 17 is a box diagram showing the electrical connections and the electronic connections of the system of the present invention with an integrated touchscreen, wherein the electrical connections are shown in solid lines, and wherein the electronic connections are shown in dashed lines.

### DETAILED DESCRIPTION OF THE INVENTION

All illustrations of the drawings are for the purpose of describing selected versions of the present invention and are not intended to limit the scope of the present invention.

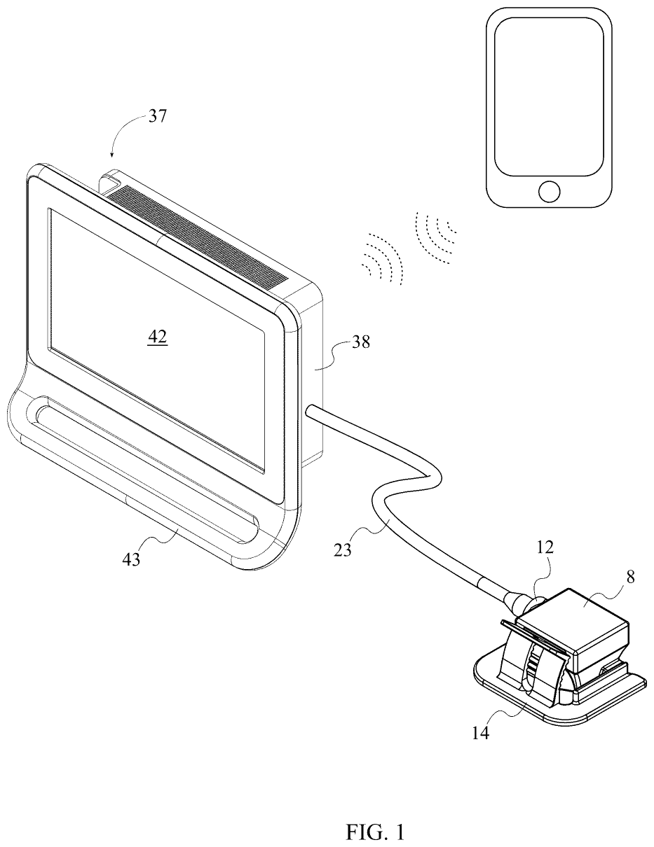

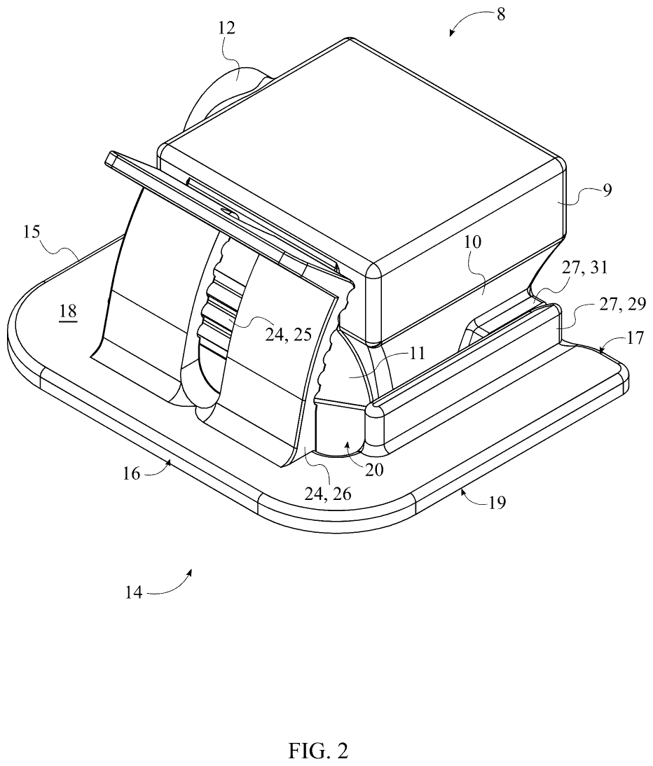

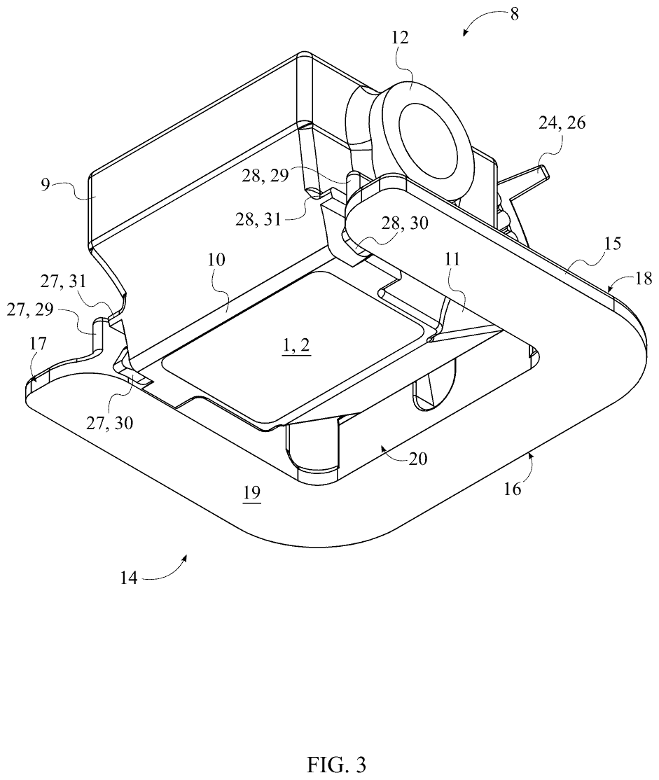

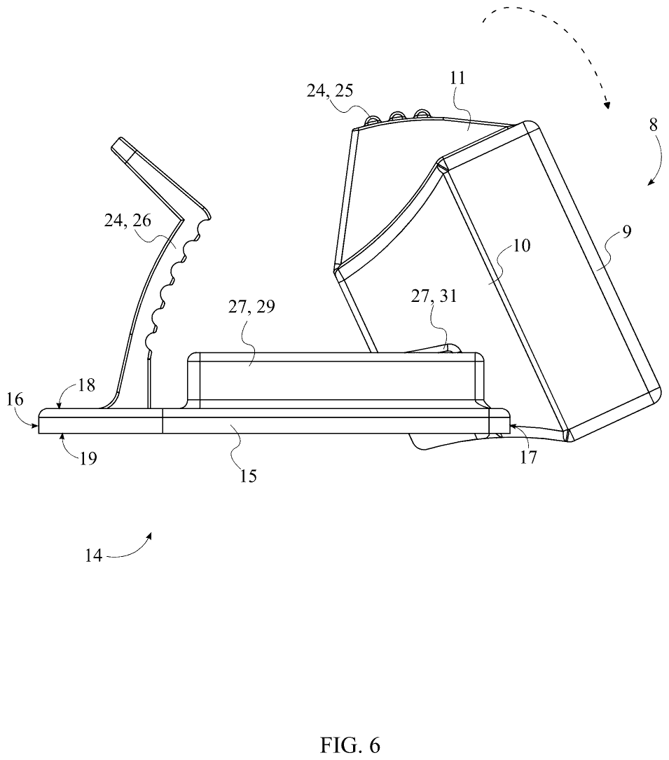

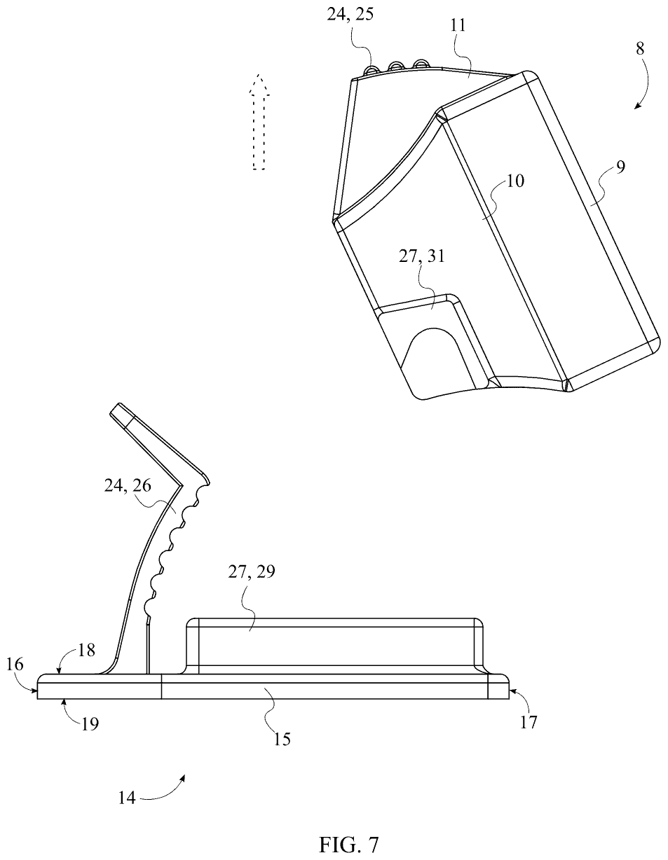

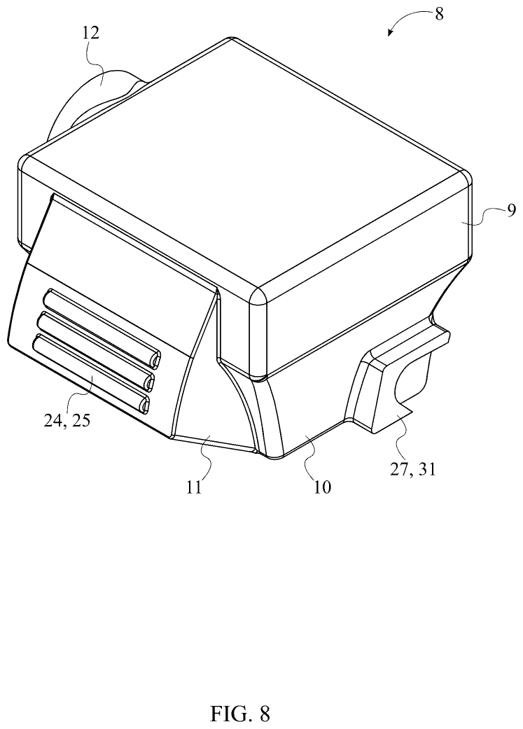

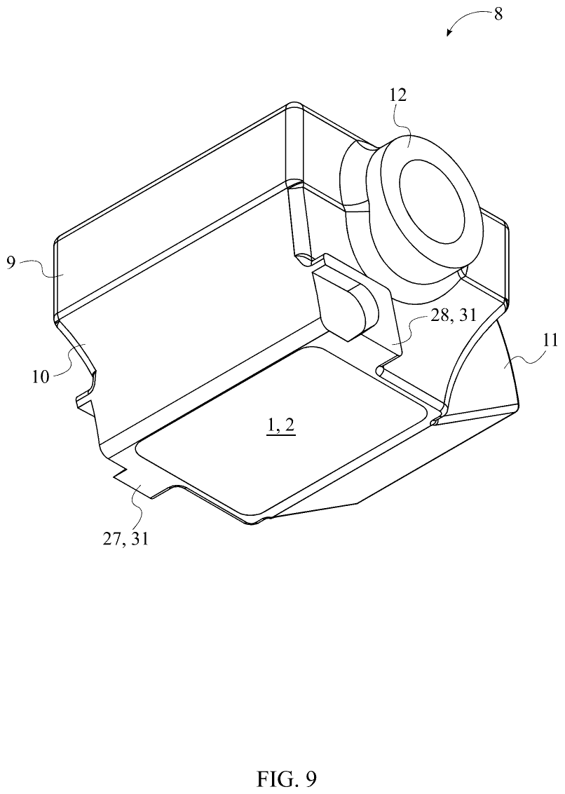

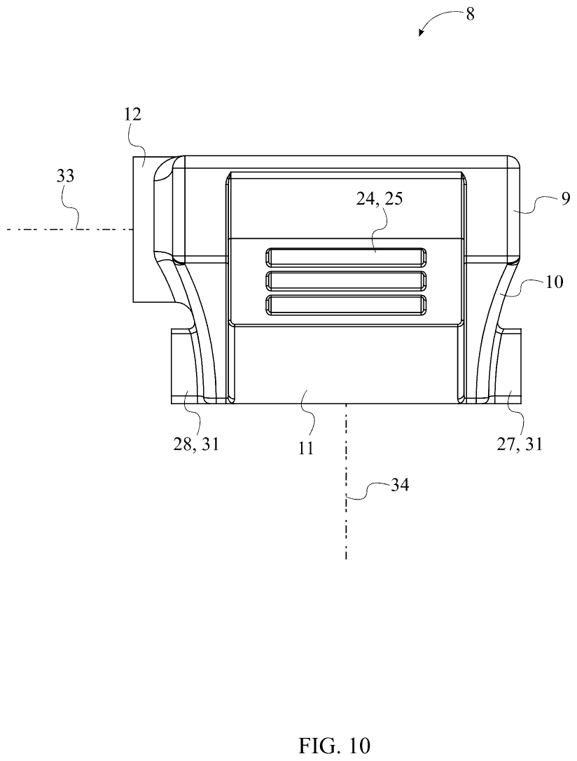

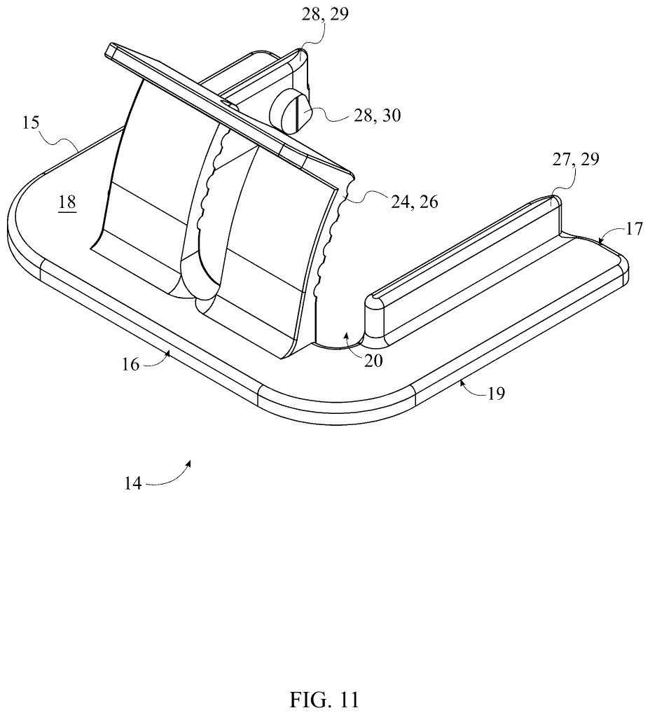

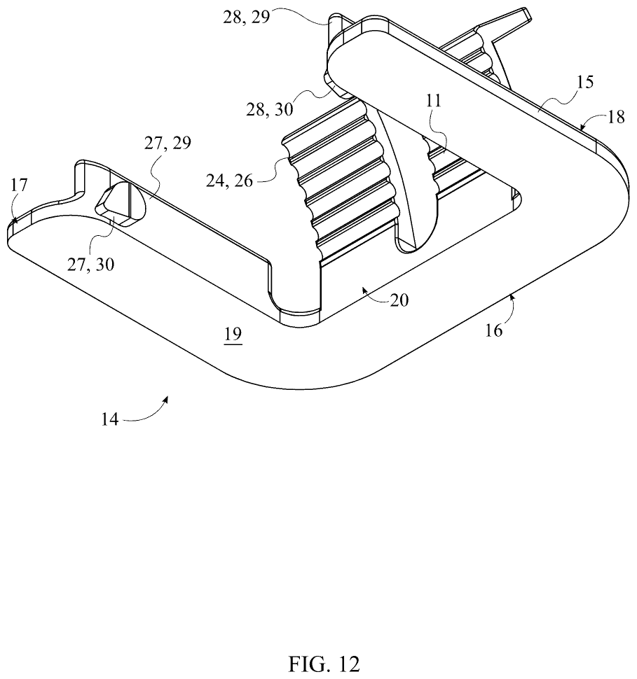

The present invention discloses a system and method for bedside cardiac ultrasound monitoring. The present invention enables the bedside monitoring of a cardiac ultrasound by a healthcare provider in a hospital setting. As can be seen in FIGS. 1 through 15, the present invention preferably comprises an ultrasonic transducer 1, a transducer housing 8, a bedside display system 37, an interface 14, at least one data/power cable 23, and a ratchet mechanism 24. The interface 14 enables the removable attachment of the transducer housing 8 to a patient's body adjacent to the target examination area. The transducer housing 8 supports the operation of the ultrasonic transducer 1 while protecting the electronics of the ultrasonic transducer 1. The ratchet mechanism 24 facilitates the adjustable coupling of the transducer housing 8 to the interface 14.

Further, the ultrasonic transducer 1 is a portable medical probe able to perform the cardiac ultrasound, as can be seen in FIGS. 1 through 15. The at least one data/power cable 23 enables the direct connection of the ultrasonic transducer 1 to the bedside display system 37, and the bedside display system 37 is preferably a computerized monitor that is situated next to a patient's hospital bed. The bedside display system 37 comprises a display housing 38, a display controller 39, and a display power source 40, which function together as a data acquisition system for the present invention. The display controller 39 is a computing unit that manages the electronic functionalities of the bedside display system 37 and that processes data captured by the ultrasonic transducer 1. The display power source 40 provides the necessary electrical power for operating the electrical and electronic components of the present invention. The display housing 38 is used as an enclosure for the internal components of the bedside display system 37 and allows the bedside display system 37 to be handled and moved by a healthcare provider.

The general configuration of the aforementioned components enables the bedside monitoring of a medical ultrasound without continuous handling by a healthcare provider. As can be seen in FIGS. 1 through 15, the transducer housing 8 is designed to be removably coupled to the interface 14 by a patient or a healthcare provider in an easy manner without much practical experience and without the use of external tools. The transducer housing 8 can have different structural shapes and sizes depending on the ultrasonic transducer 1 being implemented. The transducer housing 8 comprises an upper housing base 9 and a lower housing base 10, which are opposing bases of the transducer housing 8.

The present invention can be implemented as follows: the ultrasonic transducer 1 is mounted within the transducer housing 8, adjacent to the lower housing base 10, as can be seen in FIGS. 1 through 15. The lower housing base 10 is designed to orient the ultrasonic transducer 1 towards the target body area to be examined. Moreover, the display controller 39 is mounted within the display housing 38, while the display power source 40 is integrated into the display housing 38 so that the display housing 38 is able to act as a structural base for the display controller 39 and the display power source 40. The shape and size of the display housing 38 may depend on the system's specifications. For example, the display housing 13 can have an overall flat-screen-monitor shape large enough to accommodate the electrical and electronic components of the bedside display system 37. In preferred embodiment, the overall structure may be adapted for use with one or more mounting systems (including but not limited to VESA or other typical display mounting standards known to one of skill in the art). In another embodiment, the structure may be adapted to be mounted on an arm or boom, which may be adjustable or fixed, which is connected to a wall or ceiling of a clinical area. In another embodiment, the overall structural design may be configured such that it is portable. In a further embodiment, the structure may be adapted to be mounted to a stationary or wheeled cart, or alternately, the structure may be capable of being freestanding on a counter or tabletop.

Further, at least one data/power cable 23 is provided. The at least one data/power cable 23 is configured to tether and/or physically secure the display housing 37 to the transducer housing 8, as can be seen in FIGS. 1 through 15. In an exemplary embodiment, the at least one data/power cable 23 may be a coaxial cable or other cable, which is capable of transmitting electrical and data signals known to one having skill in the art that physically connects the display housing 37 to the transducer housing 8 while enabling the necessary electrical and electronic connections to be formed. In addition, the transducer housing 8 can be hingedly connected to the interface 14 to secure the transducer housing 8 to the interface 14. The interface 14 is preferably a flat rigid body, but the interface 14 can alternatively be shaped to fit a specific contour of a patient's body. The ratchet mechanism 24 is also operatively coupled between the interface 14 and the transducer housing 8. The ratchet mechanism 24 is used to incrementally orient the transducer housing 8 at a specific angle with the interface 14. In other words, a patient or a healthcare provider can adjust the orientation of the ultrasonic transducer 1 by changing the angle between the transducer housing 8 and the interface 14. The adjustment of orientation may be used to adapt to patient anatomy differences or ensure appropriate imaging quality is maintained for the duration of the study. In a preferred embodiment, such adjustment may be performed while the interface 14 remains attached to the patient.

Further, the display power source 40 is electrically connected to the display controller 39 to provide the necessary electrical power for operating the display controller 39, as can be seen in FIGS. 1 through 15. The display power source 22 is preferably a power cord connected to an electrical outlet. The display power source 22 can alternatively be one or more portable batteries mounted within the display housing 38. The portable batteries can be rechargeable batteries that can be recharged via a charging port on the display housing 40 or can be disposable batteries that can be replaced once empty. The type and capacity of the display power source 40 depends on the overall operational specifications of the present invention. Moreover, the display controller 39 is electronically connected to the ultrasonic transducer 1 by the at least one data/power cable 23 in order to relay digital signals between the display controller 39 and the ultrasonic transducer 1. Furthermore, the display power source 40 is electrically connected to the ultrasonic transducer 1 by the at least one data/power cable 23 to provide the necessary electrical power for operating the ultrasonic transducer 1.

As previously discussed, the interface 14 enables the removable attachment of the transducer housing 8 to a patient's body. As can be seen in FIGS. 1 through 13, the interface 14 is preferably designed to securely attach to a patient's body by a healthcare provider so that the ultrasonic transducer 1 is correctly positioned over the target body area during the ultrasound procedure. In general, the interface 14 comprises an interface base 15 and a transducer-receiving hole 20. The interface base 15 corresponds to the main structure of the interface 14, while the transducer-receiving hole 20 corresponds to the space on the interface 14 that accommodates at least a portion of the transducer housing 8, and particularly the ultrasonic transducer 1. In addition, the interface base 15 comprises a proximal base edge 16, a distal base edge 17, an upper base face 18, and a lower base face 19. The proximal base edge 16 and the distal base edge 17 correspond to two opposite edges of the interface base 15. Similarly, the upper base face 18 and the lower base face 19 correspond to the two largest opposite flat surfaces of the interface base 15.

In general, the interface 14 is implemented as follows: the proximal base edge 16 is positioned opposite the distal base edge 17 across the interface base 15, as can be seen in FIGS. 1 through 13. The interface base 15 is preferably an overall rectangular flat structure, so the proximal base edge 16 and the distal base edge 17 can be the two longest edges of the interface base 15. Further, the upper base face 18 is positioned opposite the lower base face 19 about the interface base 15 due to the flat structural design of the interface base 15. Moreover, the transducer-receiving hole 20 traverses from the upper base face 18, through the interface base 15, and to the lower base face 19, to form a space large enough to allow portions of the transducer housing 8 to pass through the interface base 15. For example, the lower housing base 10 can be positioned through the transducer-receiving hole 20 as the ultrasonic transducer 1 is positioned adjacent to the lower housing base 10.

Further, the transducer-receiving hole 20 also traverses from the distal base edge 17 and into the interface base 15 to form a fork-like design, as can be seen in FIGS. 1 through 13. So, when the transducer housing 8 is coupled to the interface 14, the transducer housing 8 is aligned with the transducer-receiving hole 20. In a preferred embodiment, at least a portion of the transducer housing 8 (or more particularly the lower housing base 10) is configured such that its shape substantially matches the general shape formed by the transducer-receiving hole 20. In a further preferred embodiment, the transducer-receiving hole 20 is generally a rectangular hole, and the lower housing base 10 also has rectangular design which allows the lower housing base 10 to move through the transducer-receiving hole 20. Furthermore, the transducer housing 8 is hingedly mounted to the upper base face 18, adjacent to the distal base edge 17 so that the angular orientation can be adjusted using the ratchet mechanism 24. In other embodiments, the interface 14 can be modified to accommodate different designs of the transducer housing 8.

As previously discussed, the ratchet mechanism 24 allows the patient to adjust the angular orientation of the transducer housing 8 on the interface 14. In the preferred embodiment, the ratchet mechanism 24 may comprise a ratchet pawl 25 and a ratchet rack 26 corresponding to the common parts of a ratchet mechanism 24, as can be seen in FIGS. 1 through 13. In addition, the transducer housing 8 may further comprise a slanted protrusion 11 that helps orient the ratchet pawl 25 towards the ratchet rack 26 when coupled together.

As can be seen in FIGS. 1 through 13, in an exemplary embodiment, the ratchet mechanism 24 can be implemented on the interface 14 as follows: the ratchet rack 26 is connected onto the upper base face 18, adjacent to the proximal base edge 16, to secure the ratchet rack 26 to the interface base 15. The ratchet rack 26 is preferably a curved thin rack that can be pulled to engage/disengage the ratchet mechanism 24. In addition, the ratchet rack 26 is oriented towards the distal base edge 17 so that the ratchet rack 26 engages the ratchet pawl 25 on the transducer housing 8. Further, the slanted protrusion 11 is laterally connected to the transducer housing 8 in between the upper housing base 9 and the lower housing base 10. This forms a curved lateral protrusion on the side of the transducer housing 8 that matches the curvature of the ratchet rack 26. Further, the slanted protrusion 11 is positioned adjacent to the ratchet rack 26 to allow the ratchet rack 26 to engage the ratchet pawl 25. The ratchet pawl 25 is connected onto the slanted protrusion 11, offset from the transducer housing 8, to secure the ratchet pawl 25 to the transducer housing 8. In addition, the ratchet pawl 25 is oriented towards the ratchet rack 26 to ensure that the ratchet pawl 25 engages the ratchet rack 26. Furthermore, the ratchet pawl 25 is selectively engaged with the ratchet rack 26 to adjust the angular orientation of the transducer housing 8 with the interface base 15.

As previously discussed, the hinged connection between the transducer housing 8 and the interface 14 allows the adjustment of the angular orientation of the transducer housing 8 using the ratchet mechanism 24, as can be seen in FIGS. 1 through 13. In addition, the hinged connection is designed to enable the removable coupling of the transducer housing 8 to the interface 14. To do so, the present invention may further comprise a first hinge mechanism 27 and a second hinge mechanism 28 that allow the removable coupling of the transducer housing 8 to the interface 14. In addition, the first hinge mechanism 27 and the second hinge mechanism 28 each comprise a flat protrusion 29, a hinge pin 30, and a pin hook 31 that enable the implementation of each hinge mechanism on the transducer housing 8 and the interface 14.

As can be seen in FIGS. 1 through 13, the first hinge mechanism 27 and the second hinge mechanism 28 can be implemented as follows: the first hinge mechanism 27 and the second hinge mechanism 28 are positioned opposite to each other about the transducer housing 8 to ensure that the transducer housing 8 can be securely coupled to the interface 14. Further, the flat protrusion 29 of the first hinge mechanism 27 is connected onto the upper base face 18, adjacent to the transducer-receiving hole 20. Similarly, the flat protrusion 29 of the second hinge mechanism 28 is connected onto the upper base face 18, adjacent to the transducer-receiving hole 20. Each flat protrusion 29 is preferably oriented perpendicular to the upper base face 18 to surround the transducer housing 8 on both sides.

Further, each hinge pin 30 is connected onto the corresponding flat protrusion 29 to incorporate the hinge pin 30 on the lateral side of the flat protrusion 29, as can be seen in FIGS. 1 through 13. The hinge pin 30 of the first hinge mechanism 27 and the hinge pin 30 of the second hinge mechanism 28 are also oriented towards each other so that each hinge pin 30 is oriented towards the transducer-receiving hole 20. Moreover, the hinge pin 30 of the first hinge mechanism 27 and the hinge pin 30 of the second hinge mechanism 28 are concentrically positioned to each other to evenly position the transducer housing 8 on the interface 14. Similarly, the pin hook 31 of the first hinge mechanism 27 and the pin hook 31 of the second hinge mechanism 28 are laterally integrated into the transducer housing 8, adjacent to the lower housing base 10. This way, the position and orientation of each pin hook 31 matches the position and orientation of the corresponding hinge pin 30. Furthermore, to hingedly connect the transducer housing 8 to the interface 14, each hinge pin 30 is rotatably attached into the corresponding pin hook 31. In other embodiments, different hinge mechanisms can be implemented that allow the removable coupling of the transducer housing 8 to the interface 14.

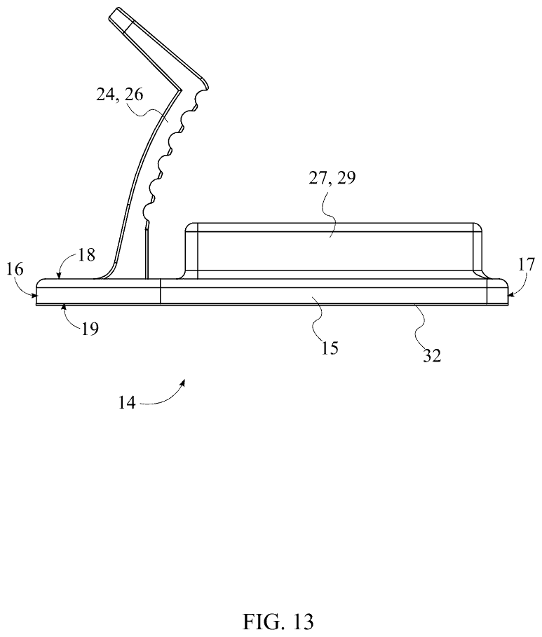

As previously discussed, the interface 14 is attached to a patient's body by a healthcare practitioner to ensure that the ultrasonic transducer 1 is always oriented towards the target body area. As can be seen in FIG. 13, to facilitate the safe attachment of the interface 14 to the patient's body, the present invention may further comprise a quantity of adhesive 32 that is safe to use on the skin. For example, the quantity of adhesive 32 can be a body adhesive or a length of double-sided tape that is safe to use on the skin. In a further embodiment, the quantity of adhesive 32 is superimposed across the lower base face 19 so that the lower base face 19 can be safely attached to the target body area. In some embodiments, a base cover can be provided that encloses the interface 14 when not in use. The base cover enables the interface 14 to be comfortably worn under clothing so that the patient does not remove the interface 14.

The transducer housing 8 is preferably tethered to the electronics housing 13 using the at least one data/power cable 23 for ease of use and to prevent misplacing either component. As can be seen in FIGS. 1 through 15, to ensure that the at least one data/power cable 23 does not obstruct the operation of the ultrasonic transducer 1, the transducer housing 8 may further comprise a cable port 12 that orients the at least one data/power cable 23 away from the ultrasonic transducer 1. To do so, the cable port 12 is laterally integrated into the transducer housing 8, adjacent to the upper housing base 9, to position the cable port 12 away from the ultrasonic transducer 1. Further, a central axis 33 of the cable port 12 is positioned perpendicular to an emission axis 34 of the ultrasonic transducer 1. This way, the cable port 12 ensures that the at least one data/power cable 23 does not compromise the orientation of the transducer housing 8 on the interface 14. Furthermore, the at least one data/power cable 23 is terminally attached into the cable port 12 to secure the at least one data/power cable 23 to the cable port 12.

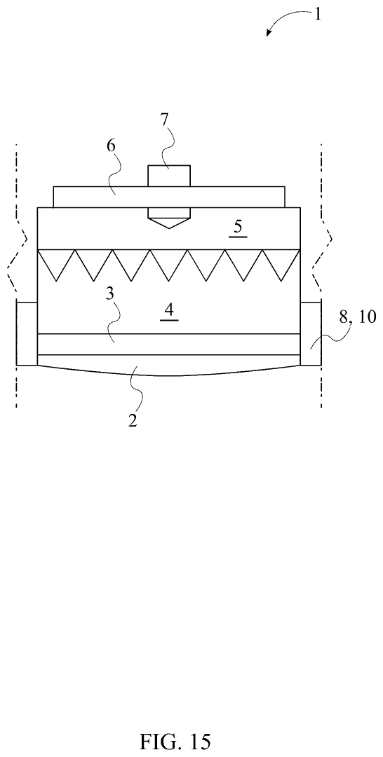

Different ultrasound technologies can be implemented for the ultrasonic transducer 1 according to the system's specifications. In general, the ultrasonic transducer 1 comprises a footprint 2, a piezoelectric crystal arrangement 3, a backing 4, a stiffener 5, a flex circuit 6, and a connector 7, as can be seen in FIG. 15. The footprint 2 corresponds to the structure of the ultrasonic transducer 1 that comes into contact with the patient's body. The piezoelectric crystal arrangement 3 converts electrical energy into sound waves and vice versa. The backing 4 and stiffener 5 helps dampen the generated sound waves to improve image quality. The flex circuit 6 enables the automatic operation of the ultrasonic transducer 1, and the connector 7 allows the electrical and electronic connection of the flex circuit 6 to the portable power source 22 and the controller 21, respectively.

In general, the ultrasonic transducer 1 can be implemented as follows: the footprint 2 is integrated into the lower housing base 10 so that the orientation of the ultrasonic transducer 1 matches the orientation of the lower housing base 10, as can be seen in FIG. 15. Further, the piezoelectric crystal arrangement 3 is mounted within the transducer housing 8, adjacent to the footprint 2, so that the piezoelectric crystal arrangement 3 is protected by the transducer housing 8. The backing 4 is mounted across the piezoelectric crystal arrangement 3, opposite to the footprint 2. In addition, the stiffener 5 is mounted across the backing 4, opposite to the piezoelectric crystal arrangement 3. This forms a layered arrangement for the piezoelectric crystal arrangement 3, the backing 4, and the stiffener 5. Further, the flex circuit 6 is mounted across the stiffener 5, opposite to the backing 4, so secure the flex circuit 6 to the stiffener 5. An additional stiffener 5 for the flex circuit 6 can implemented to support the flex circuit 6.

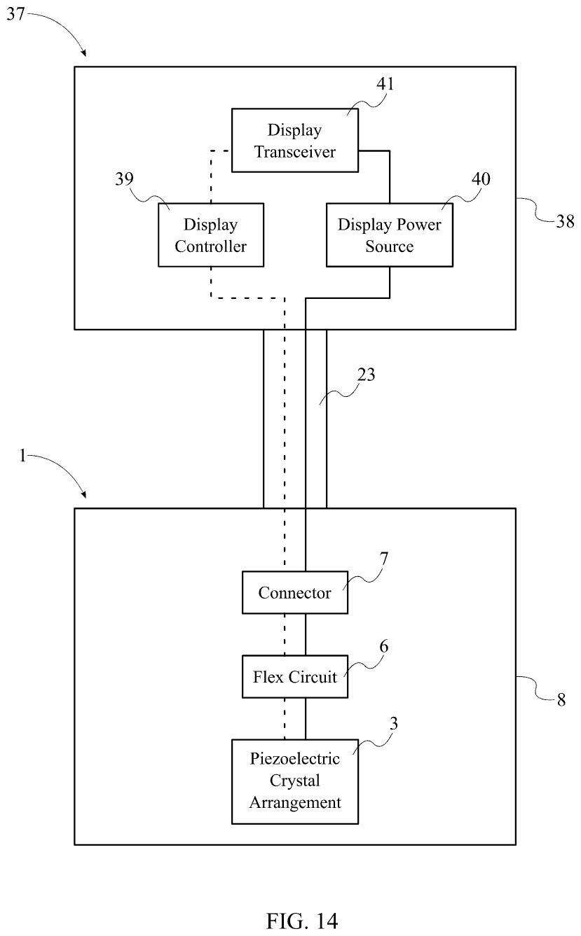

In addition, the connector 7 is mounted onto the flex circuit 6, opposite the stiffener 5, to secure the connector 7 to the flex circuit 6, as can be seen in FIGS. 14, 15, and 17. Moreover, the flex circuit 6 is electronically and electrically connected to the piezoelectric crystal arrangement 3 and the connector 7 to allow the transmission of data signals and electricity between the components. Further, the display controller 39 is electronically connected to the connector 7 by the at least one data/power cable 23 to enable the transmission of data signals between the display controller 39 and the flex circuit 6. The display power source 40 is also electrically connected to the connector 7 by the at least one data/power cable 23 to enable the transmission of electricity between the display power source 40 and the flex circuit 6. In other embodiments, different transducer technologies can be implemented as necessary.

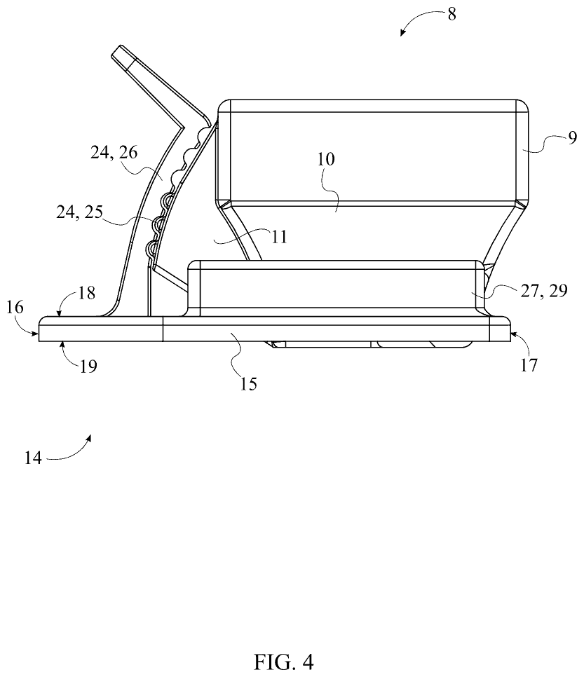

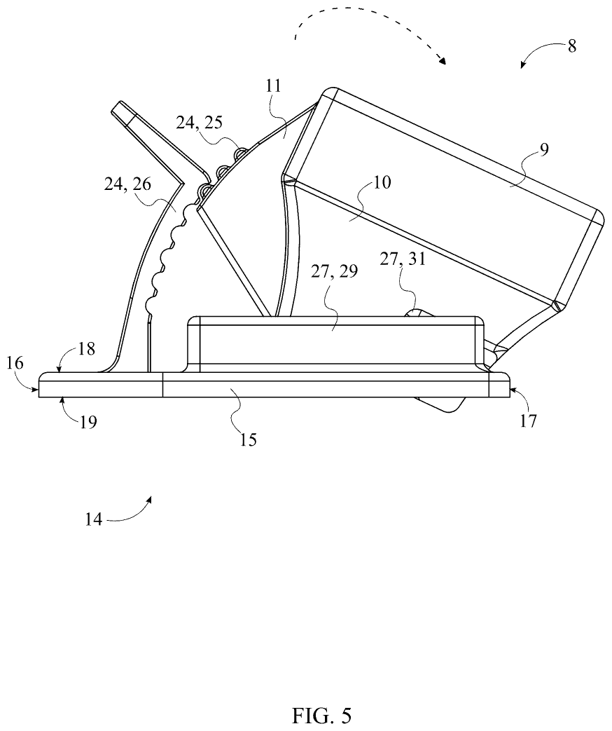

As previously discussed, the hinged connection between the transducer housing 8 and the interface 14 and the ratchet mechanism 24 allows the adjustment of the angular orientation of the transducer housing 8 on the interface 14. In the preferred embodiment, the footprint 2 and the piezoelectric crystal arrangement 3 can be oriented to the interface 14 with a projected angle set between 120 degrees to 180 degrees, as can be seen in FIGS. 4 and 5. In other embodiments, the hinge connection and/or the ratchet mechanism 24 can be modified to accommodate a greater angular degree of freedom.

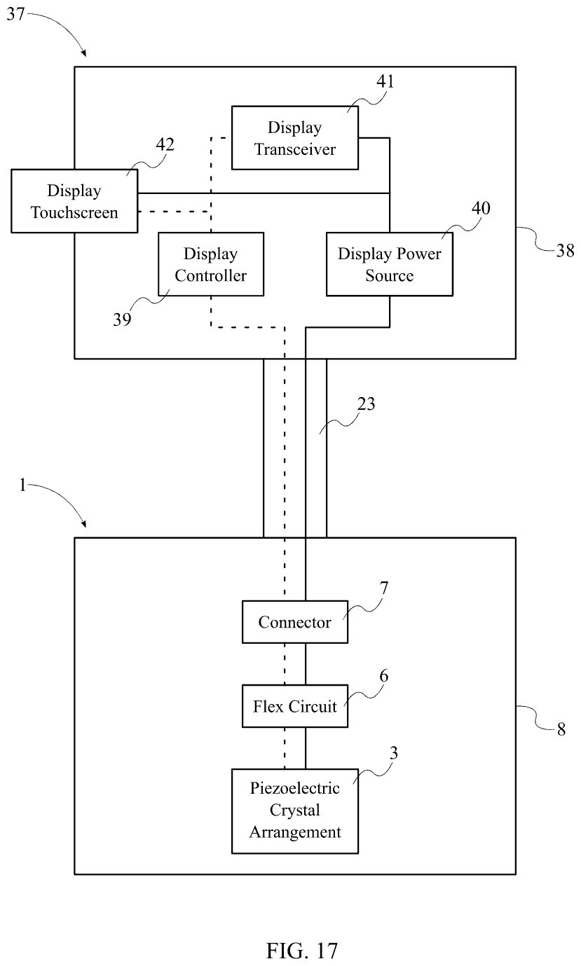

The present invention may enable the wireless transmission of the captured data to an external computing device, such as a patient's or healthcare provider's smartphone, which allows the captured data to be relayed to a healthcare database via an Internet connection. The patient or healthcare provider can then monitor and analyze the captured data from a remote location. As can be seen in FIGS. 14 and 17, to facilitate the wireless transmission of data, the bedside display system 37 may further comprise a display transceiver 41. The display transceiver 41 enables the wireless transmission of the captured data using common wireless technologies including, but not limited to, short-range radio communication technologies, long-range radio communication technologies, etc.

In the preferred embodiment, the display transceiver 41 is mounted within the display housing 38 so that the display transceiver 41 is protected by the display housing 38, as can be seen in FIGS. 14 and 17. The display controller 39 is electronically connected to the display transceiver 41 to enable the transmission of data signals between the display controller 39 and the display transceiver 41. Further, the display power source 40 is electrically connected to the display transceiver 41 to enable the transmission of electrical power from the display power source 40 to the display transceiver 41. Additional features can be implemented on the bedside display system 37. For example, a power button can be implemented to allow a patient or a healthcare provider to selectively turn the system on or off. In other embodiments, different operational features can be implemented on the bedside display system 37.

The present invention may also allow a patient or a healthcare provider to directly engage the ultrasonic transducer 1 without the use of an external computing device. As can be seen in FIGS. 1, 16 and 17, the bedside display system 37 may further comprise a display touchscreen 42 that allows a user to directly interface with the present invention. The display touchscreen 42 is externally mounted into the display housing 38 so that a patient or a healthcare provider can easily access the display touchscreen 42. In addition, the display controller 39 is electronically connected to the display touchscreen 42 to relay digital signals between the display controller 39 and the display touchscreen 42. Furthermore, the display power source 40 is electrically connected to the display touchscreen 42 to provide the necessary electrical power for operating the display touchscreen 42. In other embodiments, different computational accessories can be operatively integrated into the bedside display system 37 to functionally enhance the present invention.

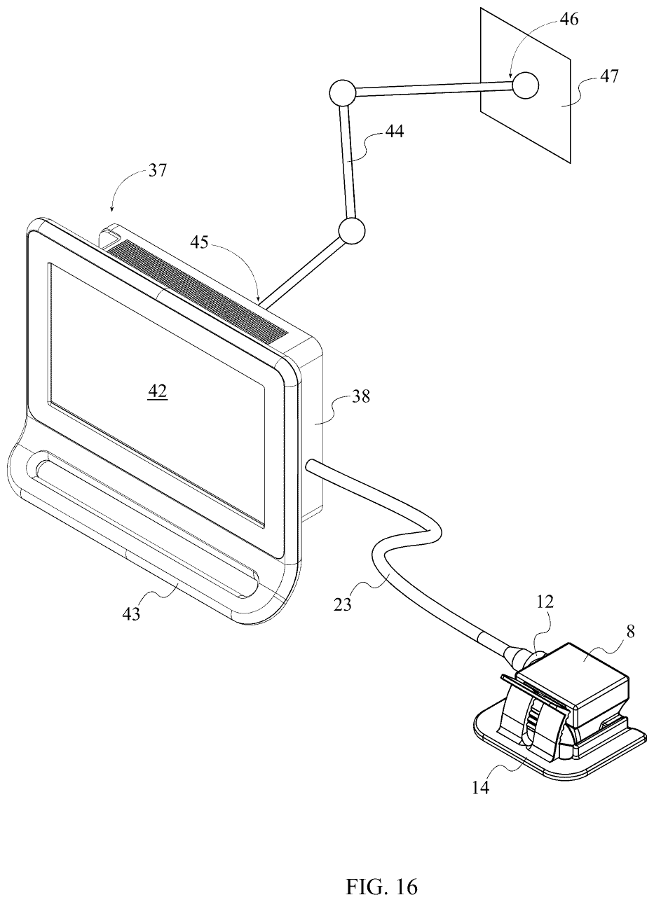

As can be seen FIGS. 1 and 16, the bedside display system 37 may further comprise a display handle 43, which allows a patient or a healthcare provider to grasp and move the bedside display system 37 to a new location and/or new orientation. Thus, the display handle 43 is externally mounted to the display housing 38 so that a patient or a healthcare provider can get a hold of the bedside display system 37 and can then reposition the bedside display system 37 for better viewing or to move the bedside display system 37 out of the way.

As can be seen FIG. 16, the bedside display system 37 may further comprise a swivel arm 44 and a wall mount 47, which are used to physically support the bedside display system 37 from a wall. The swivel arm 44 allows for some degrees of freedom in the repositioning of the bedside display system 37, while the wall mount 47 anchors the bedside display system 37 and the swivel arm 44 to a wall. The swivel arm 44 preferably has at least one mechanical joint to enable those degrees of freedom. Moreover, a proximal arm end 45 of the swivel arm 44 is pivotably mounted to the display housing 38, which allows the bedside display system 37 and the swivel arm 44 to be reoriented in relation to each other. A distal arm end 46 of the swivel arm 44 is pivotably mounted to the wall mount 47, which allows the swivel arm 44 and the wall mount 47 to be reoriented in relation to each other.

As previously discussed, an external computing device for a patient or a healthcare provider can be, but is not limited to, a smartphone or other computing devices capable of connecting to the Internet. A software application for the present invention can be developed to be compatible with different computing devices. Further, the software application is designed to utilize the Internet connectivity of the external computing device to communicate with a corresponding healthcare database that allows an authorized healthcare provider to access the collected data for remote monitoring and examination. Furthermore, the software application can be provided with different functions that enable a patient and/or a healthcare provider to control the present invention during the bedside cardiac ultrasonic procedure.

An overall process for using the present invention includes the following steps. A healthcare provider begins the overall process by laying a patient down on a flat surface such as a bed. The healthcare provider then removes the base cover of the interface 14 and applies the coupling material within the transducer-receiving hole 20. The coupling material can be, but is not limited to, ultrasonic gel applied over the patient's skin exposed within the transducer-receiving hole 20. Afterwards, the healthcare provider attaches the transducer housing 8 to the interface base 15 using the first hinge mechanism 27 and the second hinge mechanism 28. Next, the healthcare provider adjusts the position of the transducer housing 8 on the interface base 15 using the ratchet mechanism 24.

Once the ultrasonic transducer 1 is properly positioned on the interface 14, the healthcare provider can activate a software application executed by the bedside display system 37. Afterwards, the ultrasonic transducer 1 is used to generate small ultrasonic loops, which are processed and recorded by the display controller 39. The ultrasonic loops are preferably three to five seconds long as recommended by the healthcare provider. The captured data is processed and relayed to the bedside display system 37. Once the captured data is relayed to the bedside display system 37, cine-loop analysis and ventricular function data calculations are performed, preferably using Artificial Intelligence (AI) algorithms implemented on the software application executed by the bedside display system 37. Then, the analysis and calculation data is relayed to the corresponding healthcare database via the Internet connectivity of the bedside display system 37.

The healthcare provider can also access the analysis and calculation data from the corresponding healthcare database for review and evaluation. Once the bedside cardiac ultrasonic procedure is complete, the healthcare provider can turn off the bedside display monitor 37 and can remove the transducer housing 8 from the interface 14. The uncoupling process involves first disengaging the ratchet mechanism 24 by pulling the ratchet rack 26 away from the ratchet pawl 25. Then, the healthcare provider rotates the transducer housing 8 until the first hinge mechanism 27 and the second hinge mechanism 28 can also be disengaged. Then, the healthcare provider can safely remove the transducer housing 8 from the interface 14. Afterwards, the healthcare provider and/or the patient can clean the skin and cover the interface 14 with the base cover. In other embodiments, the present invention can be modified to accommodate different examination functions or other subprocess related to the bedside cardiac ultrasonic procedure.

Although the invention has been explained in relation to its preferred embodiment, it is to be understood that many other possible modifications and variations can be made without departing from the spirit and scope of the invention.

What is claimed is:

1. A system for bedside cardiac ultrasonic monitoring comprising:
an ultrasonic transducer;
a transducer housing;
a bedside display system;
an interface;
at least one data/power cable;
a ratchet mechanism;
the transducer housing comprising an upper housing base and a lower housing base;
the bedside display system comprising a display housing, a display controller, and a display power source;
the ultrasonic transducer being mounted within the transducer housing, adjacent to the lower housing base;
the display controller being mounted within the display housing;
the display power source being integrated into the display housing;
the bedside display system being tethered to the transducer housing by the at least one data/power cable;
the transducer housing being hingedly connected to the interface;
the ratchet mechanism being operatively coupled between the interface and the transducer housing, wherein the ratchet mechanism is used to incrementally orient the transducer housing at a specific angle with the interface;
the display power source being electrically connected to the display controller;
the display controller being electronically connected to the ultrasonic transducer by the at least one data/power cable; and
the display power source being electrically connected to the ultrasonic transducer by the at least one data/power cable.

2. The system for bedside cardiac ultrasonic monitoring as claimed in claim 1 further comprising:
the interface comprising an interface base and a transducer-receiving hole;
the interface base comprising a proximal base edge, a distal base edge, an upper base face, and a lower base face;
the proximal base edge being positioned opposite the distal base edge across the interface base;
the upper base face being positioned opposite the lower base face about the interface base;
the transducer-receiving hole traversing from the upper base face, through the interface base, and to the lower base face;
the transducer-receiving hole further traversing from the distal base edge and into the interface base;
the transducer housing being aligned with the transducer-receiving hole; and
the transducer housing being hingedly mounted to the upper base face, adjacent to the distal base edge.

3. The system for bedside cardiac ultrasonic monitoring as claimed in claim 2 further comprising:
the ratchet mechanism comprising a ratchet pawl and a ratchet rack;
the transducer housing further comprising a slanted protrusion;
the ratchet rack being connected onto the upper base face, adjacent to the proximal base edge;
the ratchet rack being oriented towards the distal base edge;
the slanted protrusion being laterally connected to the transducer housing in between the upper housing base and the lower housing base;
the slanted protrusion being positioned adjacent to the ratchet rack;
the ratchet pawl being connected onto the slanted protrusion, offset from the transducer housing;
the ratchet pawl being oriented towards the ratchet rack; and
the ratchet pawl being selectively engaged with the ratchet rack.

4. The system for bedside cardiac ultrasonic monitoring as claimed in claim 2 further comprising:
a first hinge mechanism;
a second hinge mechanism;
the first hinge mechanism and the second hinge mechanism each comprising a flat protrusion, a hinge pin, and a pin hook;
the first hinge mechanism and the second hinge mechanism being positioned opposite to each other about the transducer housing;
the flat protrusion of the first hinge mechanism being connected onto the upper base face, adjacent to the transducer-receiving hole;
the flat protrusion of the second hinge mechanism being connected onto the upper base face, adjacent to the transducer-receiving hole;
the hinge pin being connected onto the flat protrusion;
the hinge pin of the first hinge mechanism and the hinge pin of the second hinge mechanism being oriented towards each other;
the hinge pin of the first hinge mechanism and the hinge pin of the second hinge mechanism being concentrically positioned to each other;
the pin hook of the first hinge mechanism and the pin hook of the second hinge mechanism being laterally integrated into the transducer housing, adjacent to the lower housing base; and
the hinge pin being rotatably attached into the pin hook.

5. The system for bedside cardiac ultrasonic monitoring as claimed in claim 2 further comprising:
a quantity of adhesive; and
the quantity of adhesive being superimposed across the lower base face.

6. The system for bedside cardiac ultrasonic monitoring as claimed in claim 1 further comprising:
the transducer housing comprising a cable port;
the cable port being laterally integrated into the transducer housing, adjacent to the upper housing base;
a central axis of the cable port being positioned perpendicular to an emission axis of the ultrasonic transducer; and
the at least one data/power cable being terminally attached into the cable port.

7. The system for bedside cardiac ultrasonic monitoring as claimed in claim 1 further comprising:
the ultrasonic transducer comprising a footprint, a piezoelectric crystal arrangement, a backing, a stiffener, a flex circuit, and a connector;
the footprint being integrated into the lower housing base;
the piezoelectric crystal arrangement being mounted within the transducer housing, adjacent to the footprint;
the backing being mounted across the piezoelectric crystal arrangement, opposite to the footprint;
the stiffener being mounted across the backing, opposite to the piezoelectric crystal arrangement;
the flex circuit being mounted across the stiffener, opposite to the backing;
the connector being mounted onto the flex circuit, opposite the stiffener;
the flex circuit being electronically and electrically connected to the piezoelectric crystal arrangement and the connector;
the display controller being electronically connected to the connector by the at least one data/power cable; and
the display power source being electrically connected to the connector by the at least one data/power cable.

8. The system for bedside cardiac ultrasonic monitoring as claimed in claim 7, wherein the footprint and the piezoelectric crystal arrangement are oriented to the interface with a projected angle set between 120 degrees to 180 degrees.

9. The system for bedside cardiac ultrasonic monitoring as claimed in claim 1 further comprising:
the bedside display system further comprising a display transceiver;
the display transceiver being mounted within the display housing;
the display controller being electronically connected to the display transceiver; and
the display power source being electrically connected to the display transceiver.

10. The system for bedside cardiac ultrasonic monitoring as claimed in claim 1 further comprising:
the bedside display system further comprising a display touchscreen;
the display touchscreen being externally mounted into the display housing;
the display controller being electronically connected to the display touchscreen; and
the display power source being electrically connected to the display touchscreen.

11. The system for bedside cardiac ultrasonic monitoring as claimed in claim 1 further comprising:
the bedside display system further comprising a display handle; and
the display handle being externally mounted to the display housing.

12. The system for bedside cardiac ultrasonic monitoring as claimed in claim 1 further comprising:
the bedside display system further comprising a swivel arm and a wall mount;
the swivel arm comprising a proximal arm end and a distal arm end;
the proximal arm end being pivotably mounted to the display housing; and
the distal arm end being pivotably mounted to the wall mount.

13. The system for bedside cardiac ultrasonic monitoring as claimed in claim 1, wherein the interface is a flat rigid body.

14. The system for bedside cardiac ultrasonic monitoring as claimed in claim 1, wherein the interface is a flat rigid body.
